(19) United States
(12) Patent Application Publication
(10) Pub. No.: US 2006/0063993 A1
(43) Pub. Date: Mar. 23, 2006

(54) Method and apparatus for non-invasive measurement of blood analytes

(76) Inventors: Dejin Yu, Fremont, CA (US); Wei Yang, Fremont, CA (US)

Correspondence Address: STALLMAN & POLLOCK LLP, 353 SACRAMENTO STREET, SUITE 2200, SAN FRANCISCO, CA, 94111, US

(21) Appl. No.: 10/940,791

(22) Filed: Sep. 14, 2004

Related U.S. Application Data
(63) Continuation-in-part of application No. 10/914,761, filed on Aug. 9, 2004.

(51) Int. Cl. A61B 5/00

(52) U.S. Cl. 600/322

(57) ABSTRACT

The present invention discloses a method and apparatus and method for achieving non-invasive measurement of analytes from human and animal blood through the skin using Raman lightwave technology. The apparatus includes a hydraulic tissue permeation unit, which controls the amount of blood in the laser tissue interaction region. Two or more spectra are obtained at different blood levels. These spectra are used to improve the measurements.

### CLAIM OF PRIORITY

The present application is a continuation-in-part of U.S. patent application Ser. No. 10/914,761, filed Aug. 9, 2004, the disclosure of which is incorporated in this document by reference.

### TECHNICAL FIELD OF THE INVENTION

This invention in general relates to methods and apparatus for non-invasive measurement of the concentrations of analytes within human/animal blood through the skin, and in particular, for monitoring the blood glucose levels in vivo for diabetes using light scattering technology and calibrating the effects from skin and other surrounding tissue constituents.

### BACKGROUND OF THE INVENTION

Currently, daily blood glucose monitoring for diabetes patients can only be done through the use of invasive techniques. The invasive methods require drawing blood from patients, which is painful and inconvenient since the skin has to be lanced in order to collect the blood sample for measurement. 6-8 times a day, it is the same routine for the diabetics to prick their fingertips to produce a pinpoint-sized drop of blood. It is an unpleasant practice, but that is exactly what many diabetics have to do daily in order to measure blood glucose level to provide feedback for insulin dosing and other treatment.

Clinical research has demonstrated that frequent testing of blood glucose levels for people with diabetes results in improved disease management. Several large clinical studies have shown that tight control of blood sugar slows the progression of and development of long-term complications of diabetes, such as blindness and kidney failures. However, many people with diabetes do not test their blood glucose levels regularly due to physical pain and high material cost, as well as the risk of infections when finger was lanced. The American Diabetes Association (ADA) estimates that on average people with diagnosed diabetes only test their glucose levels slightly more than once per day. This is mainly because many barriers exist for the current monitoring methods. Accordingly, a new generation glucose monitoring device that non-invasively measures blood glucose level while providing painless and much safer sugar control is required to break down the barriers to tighten the glucose control, to counteract the progression of and development of long-term complication, and to improve the quality of life for those people who had the disease.

In the last decade, various attempts have been made to measure blood glucose level non-invasively (or in vivo), mainly using lightwave technologies in which the concentration of analytes is determined through light-matter interaction. These techniques include visible, near-infrared (IR) spectroscopy, mid-infrared (MIR) spectroscopy, infrared (IR) spectroscopy, reflectance spectroscopy, fluorescence spectroscopy, polarimetry, scatter changes, photo-acoustic spectroscopy, and Raman scattering through human eyes, etc. To date, none of these approaches has been proven to be clinically feasible. It is well known that visible and near-infrared absorption lacks the characteristic spectrum of glucose due to overtones and combination bands, leading to a flat spectrum response over this wavelength range. Further, while mid-infrared absorption detects fundamental tones of molecular vibration, the optical penetration depth over this wavelength range is extremely short, typically at the magnitude of order of the thickness of epidermis due to strong absorption of water. In recent years, the measurement of physiological glucose level using Raman spectroscopy from the aqueous humor of the eye has been researched. Unfortunately, there are some fundamental issues to be addressed: 1) laser eye safety and 2) time delay between glucose in blood and aqueous humor and correlation between ocular and artery glucose levels. These unresolved issues limit the effectiveness of this approach.

Having assessed the lightwave technologies mentioned-above, Raman scattering, discovered in 1928, also called spontaneous Raman scattering (as opposed to “stimulated Raman scattering”) has emerged as a promising technology for non-invasive measurement of blood glucose through the skin rather than from aqueous humor of eye. This is because, unlike infrared absorption, Raman scattering has “fingerprint” effect in that the scattered spectrum has a one-to-one correspondence to a scatterer molecule, such as glucose molecule. For a review and technical problems of some early work, see U.S. Pat. No. 5,553,616 by F. M. Ham et al. A. J. Berger et al. (U.S. Pat. No. 5,615,673) which described a method based on Raman spectroscopy for analysis of blood gases. Together with other inventions based on Raman scattering, these methods experience the following problems: 1) Raman scattering is quite weak, 2) biological effects from heart pulses, respiration, and body movement, etc., degrade measurement, and 3) calibration against that portion of the optical response caused by the skin and other tissue substances is difficult. The last issue is critical because the amounts of protein, fats, water, etc. In different people and different skin surface conditions such as oily and turbid fingers will seriously degrade the measurement results if not properly calibrated out.

In one of Wei Yang and Shu Zhang's inventions (U.S. Pat. No. 6,167,290), which is incorporated herein by reference, the first two problems are addressed by using a negative pressure system that can increases amount of blood to be detected and hold local tissue stationery. An improvement to this negative pressure system is disclosed herein. The subject disclosure also includes improved approaches for calibrating the blood glucose measurement against surrounding substances. The method of the present invention provides a means for continuous monitoring blood glucose level, facilitating a glucose tolerance test.

Other documents of interest include U.S. Pat. No. 6,044,285, inventors of J. Chaiken and C. M. Peterson; U.S. Pat. No. 6,151,522, inventors of R. R. Alfano and W. Wang.

### SUMMARY OF THE INVENTION

This invention generally provides a method and apparatus for non-invasively measuring concentrations of analytes, preferably glucose and cholesterol but not limited thereto, from human and animal blood through the skin using a Raman lightwave technique.

It is an object of the present invention to provide a method and apparatus for monitoring blood glucose from human and animal objects without drawing blood.

It is another object of the present invention to provide a dynamic calibration method for measuring concentrations of analytes from human and animal blood through the skin using a Raman lightwave technique.

Another object of the present invention is to provide a data acquisition technique used for dynamic spectral calibration against the influence from other substances.

Still another object of the present invention is to provide a data analysis method in processing spectral data acquired from the apparatus for non-invasively measuring concentrations of analytes from human and animal blood through the skin.

Yet another object of the present invention is to provide a device that non-invasively measures blood glucose levels for home, office and hospital use. The data can be stored in memory and/or downloaded to personal computer.

Still another object is to provide an improved blood permeation unit.

Briefly, a preferred embodiment of the present invention includes an excitation laser source, an optical excitation unit, a Raman signal collection unit, a tissue permeation unit, a Raman spectrometer with a light detector array, and an electronic circuitry.

The excitation laser preferably operates in the wavelength between 750 and 1000 nm so that both excitation radiation and Raman scattered wavelength have a relatively lower absorption by the human skin and tissue and thus propagate in a longer distance. The laser is preferably a solid-state semiconductor diode laser, but not limited to such a laser. U.S. Pat. No. 6,167,290 disclosed an example of an optical excitation and collection means, and a Raman spectrometer equipped with charge-coupled device (CCD). The laser radiation can be coupled to and from the tissue directly by means of optics such as lens, mirrors, filters, etc., or via fiber optics.

The tissue permeation unit modulates tissue and blood locally. It will increase the blood amount at the beginning of the measurement so that it intensifies the Raman scattering and increases the signal-to-noise ratio, and then gradually decrease the local blood amount with time until blood depletion. In one embodiment, the unit may be made of a vacuum chamber with a transparent window and small opening or hole, which is connected with an electrically or manually driven vacuum pump that creates a negative air pressure inside the vacuum chamber. The pressure inside the chamber can be changed. The user's fingertip is placed on the hole to form a closed chamber. Under the negative air pressure, a substantial amount of blood is “sucked” into a small area of the human finger after finger is placed on the hole. As the time is increased, the blood amount will be decreased gradually.

In another embodiment, the air chamber is connected with a gas cylinder and a manually driven piston. The movement and position of the piston will determine the pressure inside the chamber.

In still another embodiment, the blood permeation unit is made of a liquid chamber that is connected with a fluid cylinder and an electrically or manually driven pump. When the liquid within the chamber is pumped out, the tissue exposed to the hole will be attracted inward and blood within capillary bed will be sucked to increase local density in the laser-blood interaction region.

Other mechanical methods can be also used for varying the level of blood in the region being measured. For example, a mechanical means can be used to press the finger and then slowly release the finger. Another example could include a variable pressure tourniquet that could slow or speed up blood flow to the region being measured. For commercial use, the approach used should be relatively low cost and not discomfort the patient.

According to one embodiment of the present invention, a series of Raman signals (spectra) are acquired with time. The first spectrum corresponds to the highest amount of blood created by the tissue permeation unit, the second spectrum corresponds to the second highest amount of blood, and so on. The last spectrum corresponds to the least amount of blood at the blood depletion. The time interval between two successive spectra may be constant or variable, depending on mechanism of tissue permeation and data processing algorithms. In these spectra, the Raman signals generated from skin and substances other than blood, referred to as “static” substances, will be unchanged during the tissue permeation. By contrast, the Raman scattering from analytes in blood will become weaker and weaker since the amount of blood is decreased with time. Thus the contribution from skin and substances other than blood can be calibrated out so that spectral difference between the two successive spectra will be independent of the presence of “static” substances. These differenced spectra will be fed into multivariate algorithms for analysis such as Principal Components Regression (PCR) or Partial Least Squares Regression (PLS) which compares the derived spectra to a calibration table of spectra associated with known blood concentrations.

In another preferred embodiment, the blood permeation unit is so controlled that the blood amount is increased at the beginning and then is decreased until blood depletion while keeping the target tissue area stationary and eliminating the effects from heart pulse, respiration and body movement during the data acquisition. The blood depletion is eventually accomplished due to the distributed tension around contact region between the skin and chamber material. In a preferred embodiment, after reaching its maximum level, the blood amount is decreased linearly with time. The measurement starts at the moment when the blood amount is at its maximum, from which the strongest Raman scattering from the blood analytes is substantially achieved. Over time, the signal intensity attributed from the blood will decrease gradually while the signal components arising from the surrounding tissues will remain relatively unchanged due to the effect of blood permeation. The so-acquired Raman spectra can be processed in various ways. In a preferred embodiment, the spectral data obtained at a given time is subtracted by the spectrum acquired when the blood is depleted, i.e., Rᵢₙ=Rᵢ−Rₙ with i=1, 2, 3, . . . , n−1 where Rᵢ is the Raman spectrum obtained at time tᵢ and Rn is the last Raman spectrum acquired at the blood depletion. Rₗ is the first spectrum with the strongest Raman scattering from blood substances. The direct advantage embedded in the new series of spectra over the raw data is that the spectral contributions arising from the surrounding static tissues are removed and the resulted spectra (Rᵢₙ) are dominated by the contribution from the blood.

Although it is believed preferable to begin measurements when the blood concentration in the tissue has been increased and then take additional measurements as the blood concentration is reduced, the subject invention is not so limited. More specifically, it is within the scope of the subject invention to increase, over time, the amount of the blood in the region of tissue illuminated while taking measurements.

In another embodiment, the effects from the “static” substances can be minimized by the use of a confocal optical system with a backscattering geometry. This system is designed to spatially filter out the signal components that come from sites other than focused point. For working principle of the confocal Raman spectroscopy see “Handbook of Optical Biomedical Diagnostics”, edited by Valery V. Tuchin (SPIE Press, 2002) and “Practical Raman Spectroscopy” edited by D. J. Gardiner and P. R. Graves (Springer-Verlag, 1989).

The aforementioned non-invasive blood glucose measuring method and device has many applications in blood glucose level monitoring and diagnostics. Further objects and advantages of the subject invention will be apparent from the following drawings and detailed description of the preferred embodiments.

### BRIEF DESCRIPTION OF THE DRAWINGS

These, as well as other features of the present invention, will become more apparent upon reference to the drawings wherein:

FIG. 1 is a block diagram illustrating a basic configuration of the apparatus used for non-invasive measurement of blood glucose level in accordance with the prior art.

FIG. 2 shows a schematic diagram of prior art confocal Raman scattering configuration.

FIG. 3 shows a preferred configuration of the apparatus with confocal configuration.

FIG. 4 is one embodiment of the present invention, showing blood permeation apparatus.

FIG. 5 illustrates the different shapes and geometrical sizes of the opening hole on which the human finger is placed.

FIG. 6 shows the preferred response curve of Raman intensity as a function of measurement time.

FIG. 7 illustrates the working principle of dynamic spectral calibration against “static” substances in accordance with the present invention.

FIG. 8 shows experimental spectra according to the working principle of spectral calibration against “static” substances shown in FIG. 7.

FIG. 9 shows experimental spectra according to another working principle of spectral calibration against “static” substances.

FIG. 10 describes the flow chart of preferred data analysis and signal processing.

### DETAILED DESCRIPTION OF THE PREFERRED EMBODIMENTS

The present invention provides a method and apparatus for non-invasive measurement of blood analytes with dynamic spectral calibration against the influence from skin and other tissues other than blood. The working principle is described based on Raman spectroscopy, but it can be applied to other lightwave methods including near-infrared spectroscopy, mid-infrared spectroscopy, infrared spectroscopy, reflectance spectroscopy, fluorescence spectroscopy, Fourier-transform infrared (FTIR) spectroscopy, polarization changes, scatter changes, and photo-acoustic spectroscopy.

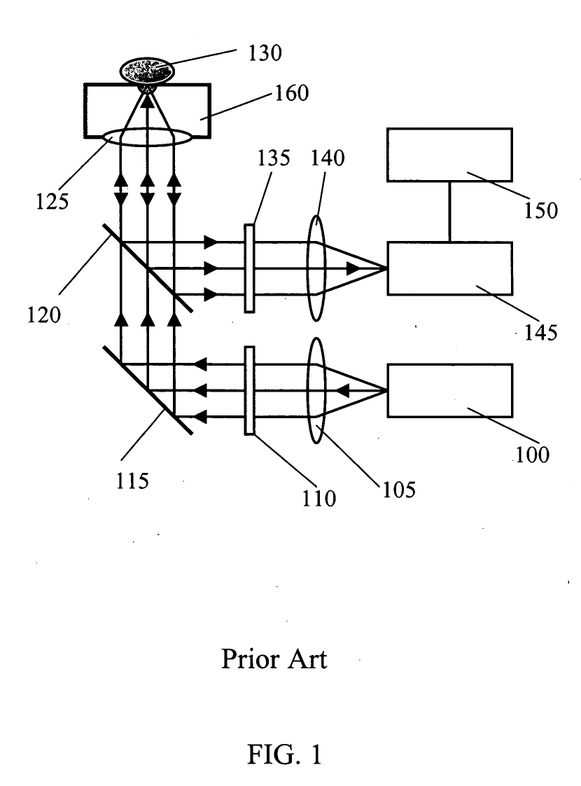

Referring now to the drawings, FIG. 1 illustrates a basic Raman configuration of the apparatus used for non-invasive measurement of blood glucose level in accordance with the prior art (U.S. Pat. No. 6,167,290). It consists of five parts: 1) excitation laser 100, 2) Raman spectrometer 145, 3) light excitation and collection unit, 4) tissue permeation unit 160, and 5) data processing unit 150. The CW excitation laser beam is generated from a laser 100, preferably semiconductor laser operated at 750-1000 nm, collimated by a lens 105, filtered by a bandpass filter 110, reflected by a mirror 115, and finally focused by a lens 125 onto the finger 130. The optical elements 100, 105, 110, 115, and 125 form the light excitation unit. The backscattered Raman light from the analytes within 130 through the skin is collected and collimated by the lens 125, reflected by the beam splitter 120, filtered by a notch filter 135 and then focused by a lens 140 onto the entrance slit of Raman spectrometer 145. The optical elements 125, 120, 135, and 140 form the light collection unit. The dispersed Raman spectra are recorded by the detector array, preferably a charge-coupled device (CCD) and transferred to the data processing unit 150 for processing and analysis. Mechanically interfaced with the analytes is the tissue permeation unit 160.

As disclosed in U.S. Pat. No. 6,167,290, a vacuum pump can be used to produce negative pressure with the chamber so that the blood within the tissue can be “sucked” toward the light-matter interaction region. The excitation laser is coupled to and the Raman signal is collected from the tissue through the lens. The tissue permeation unit 160 will increase the blood amount at the beginning of the measurement so that it intensifies the Raman scattering and increases the signal-to-noise ratio. It then gradually decreases the local blood amount with time until blood depletion. It also holds the tissue stationary to eliminate the influence from body movement, respirations, pulses, etc. Depending on the size of the hole through which the part of finger exposes to the vacuum chamber, the blood amount exhibits some functional relationship to the time. We believe that when the diameter of the hole is about 6-7 mm, the variation of the magnitude of the spectral features associated with blood constituents will be relatively linear over time.

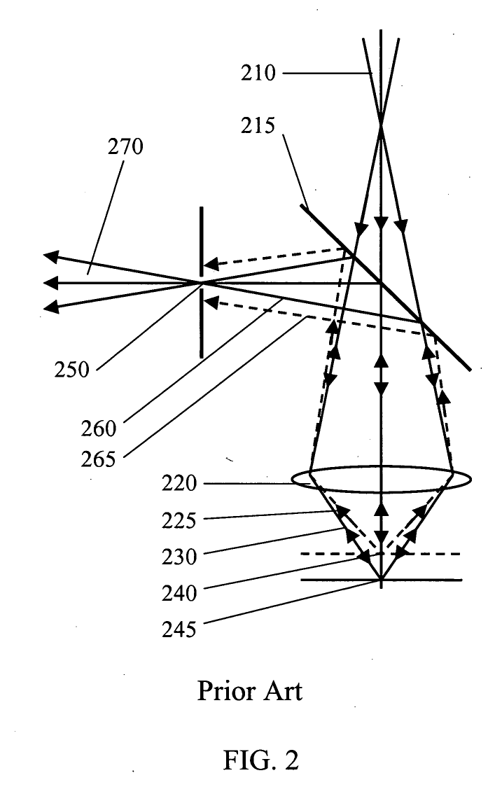

The setup shown in FIG. 1 is a backscattering configuration that is suitable for investigating absorbing samples, such as human skin that shows relatively high water absorption. The performance can be further improved by using a confocal configuration, whose principle is shown in FIG. 2. Referring to FIG. 2, in this system, a pinhole 250 (confocal hole) is used, which is at the image point of the object 245. The excitation laser beam 210 is focused by the lens 220 to the sample 245. The backscattered Raman signal 230 is collected by the same lens 220 and reflected by the beam splitter 215 to form a beam 260. Because the pinhole 250 is confocal to the point 245, the beam 260 can pass through the pinhole 250 to form the beam 270, which is further delivered to Raman spectrometer. The key to this confocal arrangement is that the pinhole will reject out-of-focus signals so as to increase signal-to-noise ratio and reduce background influence. An illustration of an out-of-focus signal is shown in FIG. 2 by dashed lines, in which a signal emitted from the point 240 is stopped by the screen and cannot pass through the pinhole 250. Further, it allows one to measure Raman spectra of analytes at different depth by adjusting the laser beam and pinhole position.

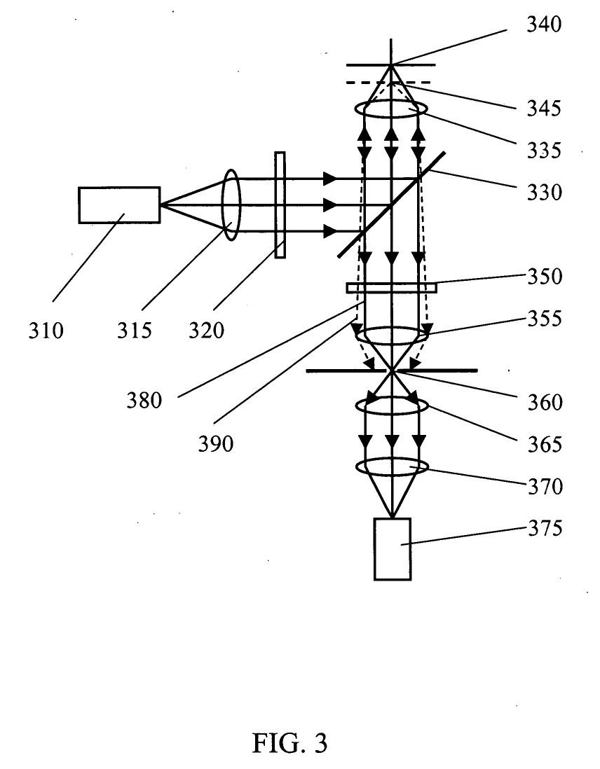

A preferred confocal configuration is illustrated in FIG. 3. The excitation laser beam generated from the laser 310 is collimated by a lens 315, filtered by a bandpass filter 320, reflected by a beam splitter 330, and focused by a lens 335 to the sample 340 to be measured. The backscattered signal from 340 is collected and collimated by the lens 335, passes through the beam splitter 330, filtered by a notch filter 350 to form the beam 380, and focused by a lens 355. This beam will pass through the pinhole 360 and is further delivered to Raman spectrometer 375, via a collimation system 365 and 370. The sample point 340 is confocal to the pinhole 360. The out-of-focus signal, such as that coming from 345 and passing through 335, 330, 350 and 355, cannot pass through the pinhole 360 and therefore is rejected.

In FIG. 1 and FIG. 3, the bandpass filters 110 and 320 allow laser wavelength to pass and block the side wavelength components while the notch filters 135 and 350 stop the signals at the laser wavelength and allow the Raman shifted signals to pass through. The preferred beam splitters 120 in FIG. 1, 215 in FIG. 2, and 330 in FIG. 3 allow 20 percent of laser power to be delivered to the sample and allow 80 percent of Raman signals to be delivered to Raman spectrometer. In another embodiment, a beam splitter that transmits laser wavelength and reflects Raman shifted wavelengths can be used in the configurations shown in FIG. 1 and FIG. 2. Similarly, a beam splitter that reflects laser wavelength and transmits Raman shifted wavelengths can be used in the configurations shown in FIG. 3.

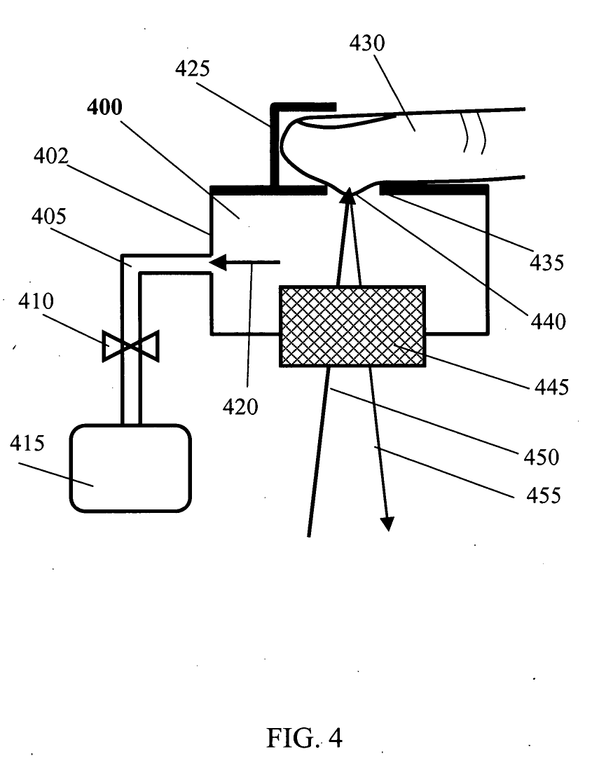

Another preferred embodiment of a tissue permeation unit 400 is illustrated in FIG. 4. Unit 400 includes a liquid chamber 402. A negative pressure is created by pumping out a small portion of the liquid 420 from the chamber 402 using a liquid pump 415. The pump rate is controlled with the valve 410. On the top of the chamber, there is an opening hole, on which the body surface, such as finger 430, is placed, forming a closed chamber. Stopper 425 holds the finger in place. Due to the negative pressure, the portion 440 of finger will be deformed and the tissue will be sucked inward. Note that the liquid should stay in contact with the skin as the negative pressure is created. The excitation laser beam 450 is coupled in and the Raman signal is coupled out through the optical window 445. When the chamber is not in use, a cover should be used to block the opening hole.

The major advantage of the liquid system over the air negative pressure system is that that light energy coupling into and out of the tissue is improved and the surface scattering reduced. This result is achieved by selecting a liquid with low absorption and a refractive index close to skin's index (index-matching). In a preferred embodiment, the index of refraction of the liquid should be in the range of 1.35 to 1.6. Water would be the least expensive, but it does have some absorbing peaks at wavelengths of interest. Other possible liquids include alcohol, acetone and methanol. Further, the miscellaneous scattering light coming from the skin surface can be largely suppressed so that the signal-to-noise ratio can be enhanced. In practice, the spacing between the optical window 445 and the portion 440 of finger should be sufficiently thin to avoid light energy loss. Suitable liquids can include water, alcohol, acetone, and methanol, etc.

The optical window 445 in FIG. 4 could be a transparent plate or a single lens in a simple case. In a preferred embodiment, the optical window 445 is an optical system consisting of a set of lenses, which increases numerical aperture and reduces image aberration. The former expands the solid angle for acceptance of Raman signal while the latter ensures the focusing position and local energy density. When a microscopic objective is used, like Raman microscope, the part of optical system has to be positioned in the chamber in order that the lens is close to the sample to be investigated.

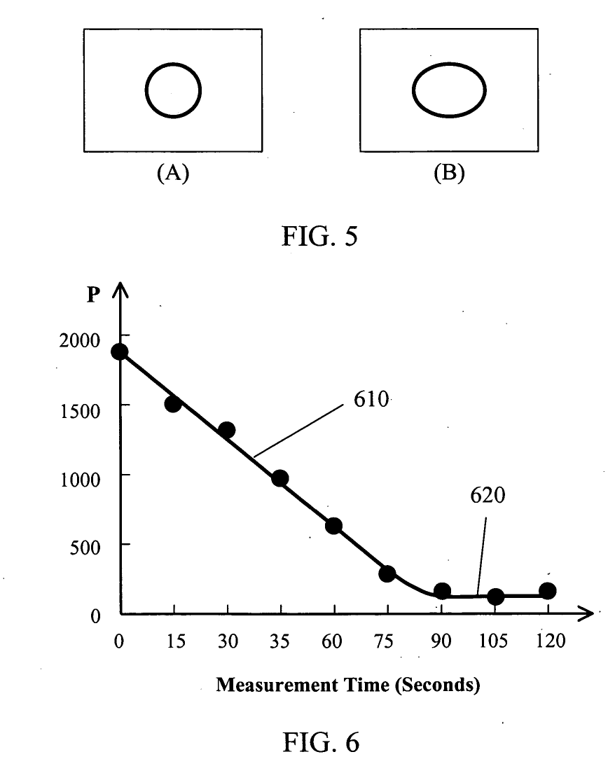

The shape and size of the opening hole 435 will have a strong effect on the blood permeation. In one of embodiments, its shape is preferably circular, as shown in FIG. 5(A). In another embodiment, the shape of the opening hole is elliptical, as shown in FIG. 5(B), to facilitate the finger. These are preferred options, but not limited to, and other shapes may be also adopted. In each type of hole, the size should be properly selected. Otherwise sufficient amount of blood would not be concentrated if the size is too small or too large.

In FIG. 4, the optical window 445 is positioned in line with the tissue. It would be possible to have the window located in another position and use a reflective surface to direct light to and from the tissue. Such an arrangement is shown in FIG. 4 of the above cited U.S. Pat. No. 6,167,290. It would also be possible to use a fiber optic element to transfer the light from the source to the tissue and back to a detector as shown in FIG. 5 of U.S. Pat. No. 6,167,290.

The permeation unit 400 can be used in various manners. In a preferred configuration, the measurements can be taken at a series of moments with an equal time interval. For example, the integration time is set 10 seconds and after 5 seconds, the next measurement is taken, as shown in FIG. 6. The level of negative pressure inside the chamber should be so controlled by the valve 410 in FIG. 4 that the Raman intensity exhibits a linear dependence 610 on the time for the first a few measurements and then quickly transitions to blood depletion state 620. The control can be incorporated in equipment calibration and implemented through a feedback loop. The blood permeation increases the blood amount in the laser-blood interaction region at the beginning, and subsequently the blood amount is linearly decreased until blood depletion in the region. The increased blood amount will intensify the Raman scattering and enhance the signal-to-noise ratio.

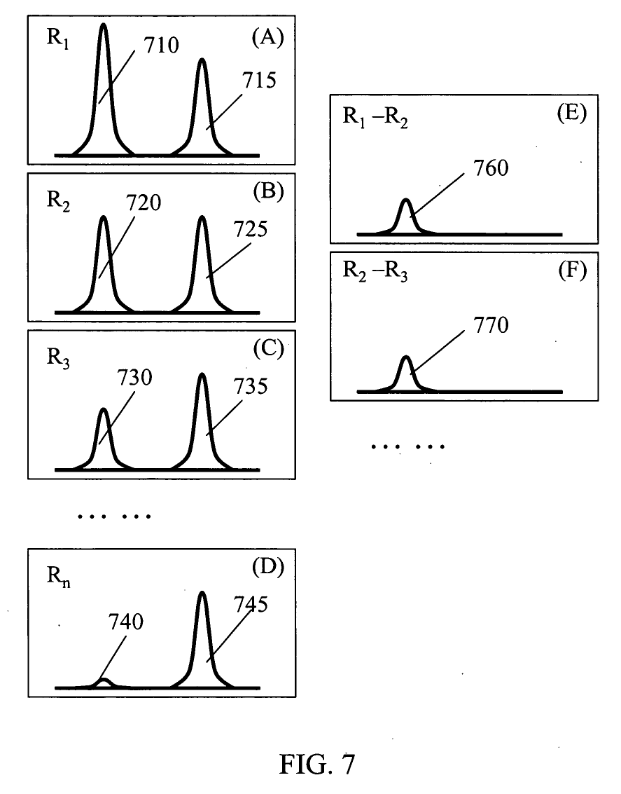

The quality and magnitude of Raman spectra collected through the apparatus shown in FIG. 3 along with the tissue permeation unit shown in FIG. 4 is greatly improved. The collected signals substantially comprise the spectral contributions from both blood and other tissues. The latter is referred as to “static” surrounding substances, which are other than substances in blood. The present invention provides a method to dynamically calibrate out spectral components coming from the static substances. In one example, a series of Raman spectra, R₁, R₂, R₃, . . . , Rₙ, are acquired with an equal time interval, as exemplified in FIG. 7 (a), (b), (c) and (d). For simplicity of description, we assume that each spectrum consists of two components: one from blood and the other from the “static” substances, such as 710 and 715 in FIG. 7 (A). As shown in these figures, the amplitude corresponding to blood analytes is decreased with time, showing a sequence from 710, 720, 730 . . . , to 740, while the amplitude associated with the static substances remains unchanged (715, 725, 735, and 745). Because the spectral contributions from the static substances are approximately constant, deriving a spectrum which represents the difference between two successive spectra will eliminate the static components. In one embodiment, the differenced spectra are calculated between two successive spectra, such as R₁-R₂ shown in FIG. 7(D). In another embodiment, discussed in greater detail below, the differenced spectra are calculated between a spectrum at any time and the spectrum at the final time. The former will give new series of spectra with approximately equal amplitude while the latter will result in spectra showing a decreasing trend. These spectra are then subject to the multivariate analysis described below.

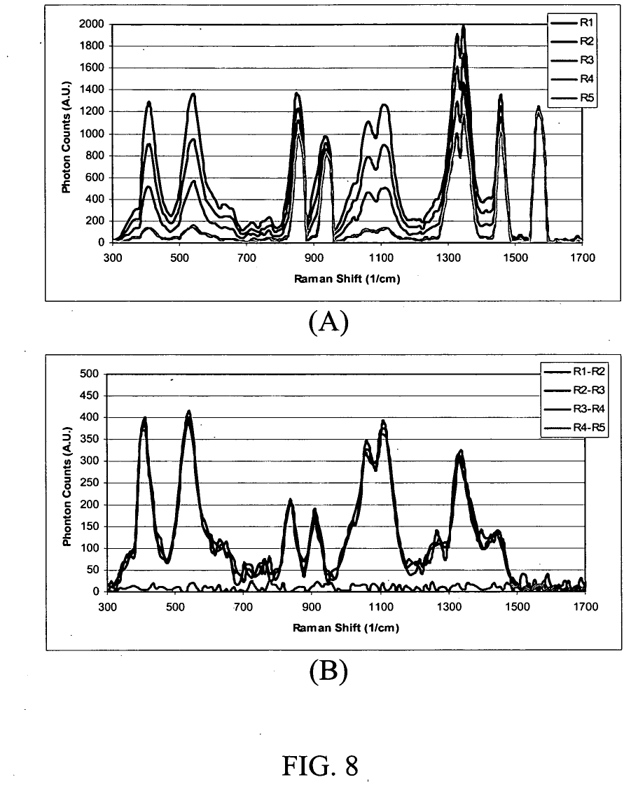

FIG. 8 shows an example of Raman spectra from a human finger and differenced signals. In FIG. 8 (A), the five raw data sets have been preprocessed to subtract background and smooth spectral fluctuation. As expected, there are three types of signals:
1) Signal amplitude changes quickly over time, such as one near 543 cm⁻¹.
2) Signal amplitude remains unchanged over time, such as one near 1568 cm⁻¹.
3) Signal amplitude change slowly over time, such as one near 938 cm⁻¹.

It is clear that the spectral contribution in the first type of signal comes from blood substances while the spectral contribution in the second type of signal originates from the static substances such as skin tissues. Finally, the spectrum in the third type is the combination of contributions from both blood and static substances. These become clearer by looking at the differenced spectra shown in FIG. 8 (B). The spectral component near 1568 cm⁻¹ in FIG. 8 (A) disappears in FIG. 8 (B). In fact, it is from amide I in human skin. The peaks at 413, 543, 1058 and 1117 cm⁻¹ change with the same rate and are associated with glucose in blood. There are four identified bands at 847, 938, 1329, and 1384 cm⁻¹, which are a combination from blood and static substances. After signal differencing, the contribution from blood is enhanced. In order to predict concentrations of some analytes, such as glucose but not limited thereto, the calculated difference spectra must be analyzed.

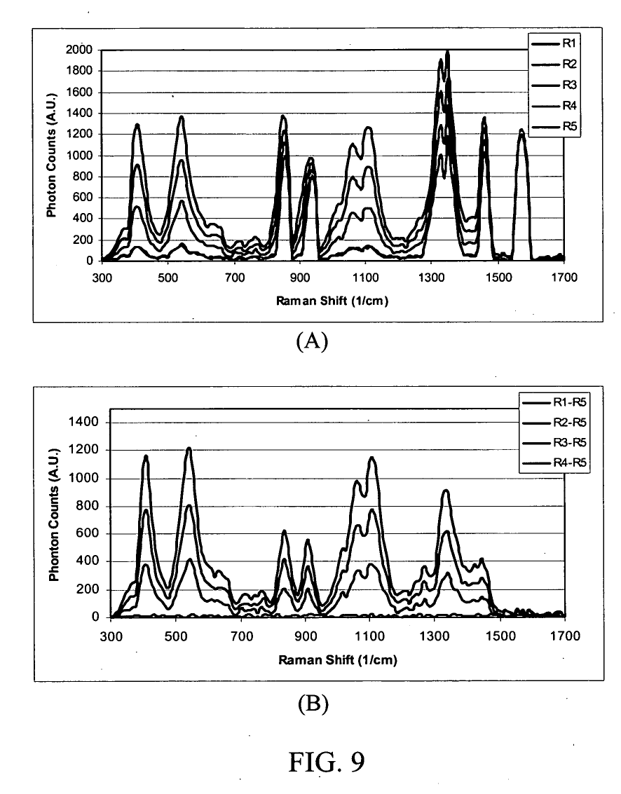

In another embodiment, an alternative data processing method is adopted to separate the two signal components responsible for blood and surrounding substances in terms of the fact that the intensity associated with blood decreases with time while the spectral contribution of the static substances is relatively unchanged with time. Thus we can differentiate the said two components by looking at the differenced signals. In one embodiment, only two spectra are acquired: the first one R₁ and the last one Rn, and one differenced spectrum R₁-Rₙ, is obtained. The model calibration and validation will rely on this differenced spectrum. In another embodiment, all differenced spectra are calculated by comparison to the last spectra when the blood is depleted, i.e., Rᵢₙ=Rᵢ−Rₙ with i=1, 2, 3, . . . , n−1 where Rᵢ is the Raman spectrum obtained at time tᵢ and Rₙ is the last Raman spectrum acquired at the blood depletion. R₁ is the first spectrum with the strongest Raman scattering from blood substances. This approach is useful to single out outliers in addition to identifying spectral contribution from the blood analytes. As an example, FIG. 9 shows the same Raman spectra as those in FIG. 8(A) from a human finger and differenced signals in FIG. 9(B) according to the signal processing described above. In order to predict concentrations of some analytes, such as glucose, the corrected signals are analyzed in the manner described above.

There are a number of well-known prior art techniques for deriving information about material constituents from a Raman spectral data. It is believed that any number of these techniques can be used. The subject approach will provide improved results because the characteristics of the derived difference spectra that are used for analysis will be dominated by blood constituents rather than being contaminated by tissue information.

Some approaches for Raman spectral analysis are set forth in the Raman Spectroscopy textbooks cited above. Further information can be found in R. L. McCreery, “Raman Spectroscopy for Chemical Analysis”, John Wiely & Sons (New York, 2000), J. R. Ferrara et al., “Introductory Raman Spectroscopy”, Academic Press (Amsterdam, 2003). See also, U.S. Pat. Nos. 5,243,983; 5,615,673 and 6,151,522, each of which are incorporated by reference herein.

In a preferred approach, a plurality of spectra are obtained from samples with known characteristics. Thus, a number of patients could be tested in a clinical trial using both the subject methodology and a suitable known invasive methodology. In this way, a table can be generated which relates the spectra measured in accordance with the subject approach to specific levels of blood constituents derived from the invasive methodology. This table can be stored. In use, one or more difference spectra on a patient with unknown blood constituents is then derived in accordance with the subject methodology. The difference spectra is compared to the stored table to determine the blood concentrations. Various well known statistical fitting and/or regression methods can be used to make this determination.

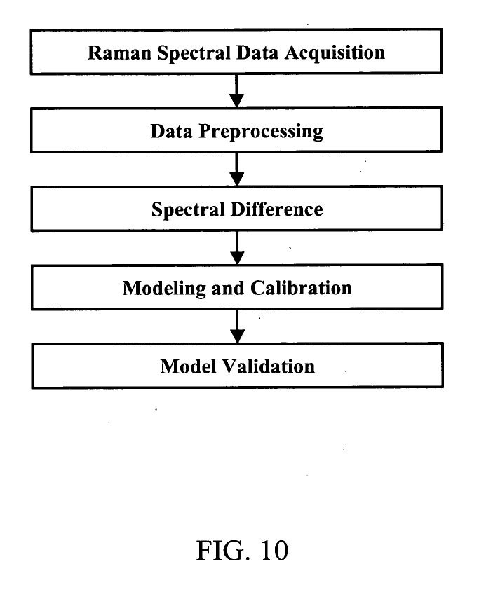

In one preferred approach, the data processing can be a multivariate analysis comprising two main steps: 1) model establishment and model validation, and 2) prediction of the concentration of analytes. A general guideline is given in FIG. 10 for analyzing the differenced spectral data obtained according to the dynamic calibration method of the present invention. First of all, a series of raw Raman spectral data for known concentrations are acquired from selected clinical specimens using the tissue permeation technique described above. The specimens should cover the full range of the concentration of interested analytes. For blood glucose measurement, the range will be from 40 mg/dL to 400 mg/dL.

Second, these spectra are preprocessed for background subtraction, spectral filtering and smoothing. Third, the data processing approach given in FIG. 8 and FIG. 9 is applied to construct a series of differenced spectra. A large portion (e.g. two-thirds) of the data will be used to establish a prediction model and the remaining data will be used to validate the model. Fourth, an appropriate prediction model is selected and established using the acquired data. For example, partial least squares regression and principal components regression methods can be adapted. These models can better cope with nonlinearity and interferences caused by other substances and instrument conditions. Finally, the model established is tested using the validation data sets.

To measure concentrations of analytes in blood of a patient, the Raman spectral data are acquired based on using the same setup as that described above. After data preprocessing and spectral difference, the data are then substituted into the validated model, from which the concentration of a blood analyte is predicted.

Although the present invention has been described in terms of specific embodiments it is anticipated that alterations and modifications thereof will no doubt become apparent to those skilled in the art. It is therefore intended that the following claims be interpreted as covering all such alterations and modifications as fall within the true spirit and scope of the invention.

We claim:

1. A non-invasive method of evaluating constituents in the blood of a patient comprising the steps of:
directing a beam of radiation to the target tissue;
measuring a first response spectrum;
modulating the target tissue to change the amount of blood in the region that is exposed to the radiation;
measuring a second response spectrum;
modulating the target tissue to change the amount of blood in the region that is exposed to the radiation;
measuring a third response spectrum;
comparing the first, second and third response spectra to identify those portions of the response most closely associated with blood in the tissue; and
predicting the constituents in the blood based on the results of the compared spectra.

2. A method as recited in claim 1, wherein the predicting step includes establishing a prediction model and validating the model based on Raman spectra associated with known levels of blood constituents.

3. A method as recited in claim 1, wherein said first, second and third response spectra are compared by performing a subtraction of magnitudes at corresponding wavelengths.

4. A method as recited in claim 1, wherein the beam of radiation is generated by a narrowband laser and the measured response spectrum corresponds to Raman Spectroscopy.

5. A method as recited in claim 1, wherein the predicting step includes comparing the results to a table of spectra associated with known levels of blood constituents.

6. A non-invasive method of evaluating constituents in the blood of a patient comprising the steps of:
applying a first negative pressure to a region of target tissue to increase the blood flow into that region;
directing optical radiation to the region;
measuring a first Raman response spectrum;
applying a second negative pressure to a region of the target tissue, said second negative pressure being different from said first negative pressure so that the blood flow in that region is changed;
measuring a second Raman response spectrum;
comparing the first and second response spectra to identify those portions of the response most closely associated with blood in the tissue; and
predicting the constituents in the blood based on the results of the compared spectra.

7. A method as recited in claim 6, wherein the predicting step includes establishing a prediction model and validating the model based on Raman spectra associated with known levels of blood constituents.

8. A method as recited in claim 6, wherein said first and second response spectra are compared by performing a subtraction of magnitudes at corresponding wavelengths.

9. A method as recited in claim 6, wherein the predicting step includes comparing the results to a table of spectra associated with known levels of blood constituents.

10. A non-invasive method of evaluating constituents in the blood of a patient comprising the steps of:
applying a first negative pressure to a region of target tissue to increase the blood flow in that region;
directing narrowband optical radiation to the region;
measuring a first Raman response spectrum;
applying a second negative pressure to the region of the target tissue, said second negative pressure being less than the first negative pressure so that blood flow in the region is reduced;
measuring a second Raman response spectrum;
subtracting the second spectrum from the first spectrum; and
predicting the constituents in the blood based on the results of the subtraction.

11. A method as recited in claim 10, wherein the predicting step includes establishing a prediction model and validating the model based on Raman spectra associated with known levels of blood constituents.

12. A method as recited in claim 10, further including the step of applying a third negative pressure to the region of the target tissue, said third negative pressure being less than the second negative pressure so that blood flow in the region is further reduced and measuring a third Raman response spectrum and wherein the third spectrum is subtracted from the second spectrum.

13. A method as recited in claim 10, wherein the predicting step includes comparing the results of the subtraction to a table of spectra associated with known levels of blood constituents

14. A non-invasive method of evaluating constituents in the blood of a patient comprising the steps of:
applying a first negative pressure to a region of target tissue to increase the blood flow in that region;
directing narrowband optical radiation to the region;
measuring a first Raman response spectrum;
applying a second negative pressure to the region of the target tissue, said second negative pressure being less than the first negative pressure so that blood flow in the region is reduced;
measuring a second Raman response spectrum;
applying a third negative pressure to the region of the target tissue, said third negative pressure being less than the second negative pressure so that blood flow in the region is reduced;
measuring a third Raman response spectrum;
subtracting the third spectrum from the first spectrum and subtracting the third spectrum from the second spectrum to obtain two difference spectra; and
evaluating the constituents in the blood based on the results of the subtractions.

15. A method as recited in claim 14, wherein the evaluating step includes comparing the results of the subtraction to a table of spectra associated with known levels of blood constituents.

16. An apparatus for non-invasively evaluating the constituents in the blood of a patient comprising:
a chamber having an opening which in use is at least partially covered by the target tissue of the patient;
a light source for directing radiation to the target tissue;
a detector for monitoring the spectral response from the target tissue and generating output signals in response thereto;
a pump for changing the pressure in the chamber in order to change the level of blood in the region of the target tissue; and
a processor for comparing the spectral responses obtained at least three different pressure levels in the chamber in order to help discriminate between the spectral response associated with the blood and the spectral response associated with the tissue, said processor further functioning to evaluate the constituents in the blood based on the results of the comparison.

17. An apparatus as recited in claim 16, wherein said chamber is filled with a fluid and said pump functions to change the amount of fluid in the chamber.

18. An apparatus for non-invasively evaluating the constituents in the blood of a patient comprising:
a chamber having an opening which in use is at least partially covered by the target tissue of the patient;
a laser light source for directing narrow band optical radiation to the target tissue;
a spectrometer detector for monitoring the Raman spectral response from the target tissue and generating output signals in response thereto;
a pump for changing the pressure in the chamber in order to change the level of blood in the region of the target tissue; and
a processor for deriving a difference spectrum by comparing the spectral responses obtained at least three different pressure levels in the chamber in order to help discriminate between the spectral response associated with the blood and the spectral response associated with the tissue, said processor further functioning to evaluate the constituents in the blood based on the results of the comparison.

19. An apparatus as recited in claim 18, wherein the spectral response of the tissue is filtered to remove wavelengths associated with the light source.

20. An apparatus as recited in claim 18, further including collection optics arranged in a confocal manner.

21. An apparatus as recited in claim 20, further including illumination optics for focusing the light onto the target tissue and additional focusing elements in the collection optics to create an image of the target tissue in a plane, and further including a confocal hole located in the image plane, said collection optics minimizing the amount of out-of-focus light reaching the detector.

22. An apparatus as recited in claim 18, wherein said chamber is filled with a fluid and said pump functions to change the amount of fluid in the chamber.

23. An apparatus for non-invasively evaluating the constituents in the blood of a patient comprising:
a laser light source for directing narrow band optical radiation to the target tissue;
a spectrometer detector for monitoring the Raman spectral response from the target tissue and generating output signals in response thereto;
means for changing the level of blood in the region of the target tissue; and
a processor for deriving a difference spectrum by comparing the spectral responses obtained at least three different levels of blood in the target tissue in order to help discriminate between the spectral response associated with the blood and the spectral response associated with the tissue, said processor further functioning to evaluate the constituents in the blood based on the results of the comparison.
